(19) United States
(12) Patent Application Publication
(10) Pub. No.: US 2008/0021276 A1
(43) Pub. Date: Jan. 24, 2008

(54) PROTECTIVE PROBE TIP, PARTICULARLY FOR USE ON A FIBER-OPTIC PROBE USED IN AN ENDOSCOPIC APPLICATION

(75) Inventor: Adam Wax, Chapel Hill, NC (US)

Correspondence Address: WITHROW & TERRANOVA, P.L.L.C., 100 REGENCY FOREST DRIVE, SUITE 160, CARY, NC, 27518, US

(73) Assignee: ONCOSCOPE, INC., Chapel Hill, NC (US)

(21) Appl. No.: 11/780,879

(22) Filed: Jul. 20, 2007

Related U.S. Application Data
(60) Provisional application No. 60/807,985, filed on Jul. 21, 2006.

(51) Int. Cl. A61B 1/00

(52) U.S. Cl. 600/122

(57) ABSTRACT

A fiber probe tip, particularly for use on a fiber-optic probe in endoscopic applications. The probe tip prevents contamination of the probe imaging elements and maintains proper distal relationships between imaging components and tissue under examination. In one embodiment, the fiber probe tip is comprised of a sheath placed over an optical fiber. The probe tip provides a sterile interface between the optical fiber and the tissue. The fiber tip probe includes an imaging element to capture reflected light from the tissue. The fiber probe tip maintains the positioning of the imaging element relative to the optical fiber to properly capture reflected light from the tissue. The fiber probe tip may also contain an optical window positioned relative to the imaging element. The optical window allows the reflected light from the tissue to pass through to the imaging element and provide an optimized focal distance between the tissue and the imaging element for the imaging technology employed.

### RELATED APPLICATION

This application claims priority to Provisional Application Ser. No. 60/807,985, entitled “Disposable, Sterile Probe Tip for Fiber Optic Probes,” filed on Jul. 21, 2006, and incorporated herein by reference in its entirety.

### FIELD OF THE INVENTION

Embodiments of the present invention relate to a probe tip for protection of a probe, including a fiber-optic probe, used in endoscope applications. The probe tip may be disposable to maintain sterility. The probe tip may also allow distance maintenance of an imaging element in the probe with respect to examined tissue to ensure the proper capture of reflected light. The probe tip may be used on a fiber-optic probe used in low coherence interferometry (LCI) endoscope applications in particular.

### BACKGROUND OF THE INVENTION

Examining tissue surfaces and/or structural features of cells in tissue is essential for many clinical and laboratory studies. For example, an endoscope is a type of probe that can be used to examine tissue surfaces. Light scattering spectrography (LSS), and low-coherence interferometry (LCI) as a method of LSS, are known techniques to allow for in vivo examination applications, including cells for determining the health status of tissues endoscopically. LSS examines variations in the elastic scattering properties of cell organelles to infer their sizes and other dimensional information. LCI has also been explored as a method of LSS. LCI utilizes a light source with low temporal coherence, wherein interference is only achieved when the path length delays of the interferometer are matched with the coherence time of the light source. For example, the inventor of the present application has developed several LCI-based techniques including an angled-resolved LCI technique in the Fourier domain (fa/LCI) to enable in vivo examination of tissue at rapid rates. This system is discussed in co-pending U.S. Patent Application Publication No. 2007/0133002 A1 (Ser. No. 11/548,468), entitled “Systems and Methods for Endoscopic Angle-Resolved Low Coherence Interferometry” (the “'468 application”), incorporated by reference herein in its entirety.

In the '468 application, an optical fiber probe is provided as one method of delivering light and collecting the angular distribution of scattered light. This example is illustrated in FIG. 1 herein and makes use of the Fourier transform properties of a lens. This property states that when an object is placed in the front focal plane of a lens, the image at the conjugate image plane is the Fourier transform of that object. The Fourier transform of a spatial distribution (object or image) is given by the distribution of spatial frequencies, which is the representation of the image's information content in terms of cycles per mm. In an optical image of elastically scattered light, the wavelength retains its fixed, original value and the spatial frequency representation is simply a scaled version of the angular distribution of scattered light.

In the fiber optic fa/LCI scheme, the angular distribution is captured by locating the distal end of the fiber bundle in a conjugate Fourier transform plane of the sample using a collecting lens. This angular distribution is then conveyed to the distal end of the fiber bundle where it is imaged using a 4f system onto the entrance slit of an imaging spectrograph. A beamsplitter is used to overlap the scattered field with a reference field prior to entering the slit so that low coherence interferometry can also be used to obtain depth resolved measurements.

Turning to FIG. 1, an example fiber-optic fa/LCI scheme is shown, which is based on a modified Mach-Zehnder interferometer. Light 10 from a broadband light source 12 is split into a reference field 14 and a signal field 16 using a fiber splitter (FS) 13. A sample probe 22 is assembled by affixing the delivery fiber 16 along the ferrule 26 at the distal end of a fiber bundle 40 such that the end face of the delivery fiber 16 is parallel to and flush with the face of the fiber bundle 40. Ball lens L1 (24) is positioned one focal length from the face of the probe 22 and centered on the fiber bundle 40, offsetting the delivery fiber 16′ from the optical axis of lens L1 (24). This configuration, which is also depicted in FIG. 2, produces a collimated beam 50 with a diameter incident on the sample 18 at an angle.

The scattered light 33 from the sample (see FIG. 2) is collected by lens L1 (24) and, via the Fourier transform property of the lens L1 (24), the angular distribution of a scattered field 36 is converted into a spatial distribution at the distal face of the multimode coherent fiber bundle 40 which is located at the Fourier image plane of lens L1 (24). The distal tip of the fiber is maintained one focal length away from lens L1 (24) to image the angular distribution of scattered light. As an illustration, the optical path of light scattered 14 at three selected scattering angles is shown in FIG. 2. In the endoscope compatible probe shown in FIG. 3, the sample 18 is located in the front focal plane of lens L1 (24) using a transparent sheath (element 58). As illustrated in FIG. 1 and also FIG. 2, scattered light emerging from a proximal end 38 of the fiber probe 26 is recollimated by lens L3 (30) and overlapped with the reference field 14 using beamsplitter BS (50). The two combined fields are re-imaged onto a slit 56 of the imaging spectrograph 54. Thus, in a fa/LCI fiber-optic probe system, it is important that the tissue of interest be located and maintained at the focal plane of the lens (e.g. L1 (24)). This is necessary to capture the angular distribution of reflected, scattered light (e.g. FIG. 2, element 33). This requires the distal end of the probe (e.g. 22) to be located approximately one focal length away from the lens (e.g. L1 (24)).

When an probe, such as probe 22 in the fa/LCI system of FIGS. 1-3, is applied endoscopically, it is typically required that sterilization be maintained during use. One method that has been developed to address this need is to provide a sheath around an existing probe. For example, U.S. Pat. No. 5,386,817 discloses a sheath around the body of an endoscope to provide sterilization. This patent includes a channel for inserting an accessory. In U.S. Pat. No. 6,863,651, the endoscope includes an endoscope with an illumination channel having a protective sheath. Both these patents include a provision for a channel through the protective sheath, which provides a point of access and thus may preclude maintenance of sterility during application of the endoscope.

An attachable channel section is disclosed as a disposable endoscope tip in U.S. Pat. No. 5,489,256 and continued in U.S. Pat. No. 5,643,175. These patents include two segments, one of which is disposable, and which are of the same cylindrical radius such that they are non-concentric. The first segment is sterilizable, and the second is disposable. The disposable section is specified to have a channel for transmitting fluid, gas, or an instrument. This channel allows potential contamination of the first segment and thus the first segment must be sterilized between uses. The configuration includes a window adjacent to an image sensor with no specification of a distance between them. Finally, this channel section is always specified with a curved surface at the end and makes no provision for a flat exterior surface.

There have also been previous devices designed specifically for protecting fiber-optic probes in particular to maintain sterilization. Specifically, U.S. Pat. No. 5,771,327 and U.S. Pat. No. 5,930,440 disclose an optical fiber probe protector consisting of a sheath with either a membrane or window at the tip. These designs specifically call for the probe to abut the protector and do not maintain a fixed distance between the probe tip and tissue sample. There are no provisions made for including an imaging optical element within the probe tip. Thus, these designs may not be used with optical based imaging systems, including but not limited to a fa/LCI system.

### SUMMARY OF THE DETAILED DESCRIPTION

In embodiments of the present invention, a new probe tip is provided to facilitate clinical application of advanced optical spectroscopic techniques when using a fiber probe or bundle while maintaining sterility. The probe tip may be used in fiber-optic probe applications. While basic optical spectroscopic techniques can be applied with a variety of configurations, newly developed advanced methods, such as the angled-resolved LCI technique in the Fourier domain (fa/LCI) system for example, require precise location of the tissue under examination relative to the optical fiber and associated imaging elements.

In embodiments of the invention, the fiber probe tip includes a protective sheath over the optical fiber or bundle. The probe tip provides a sterile interface between the optical fiber and the tissue surface under examination during endoscopic applications. Because the fiber tip probe may be employed in optical spectroscopic techniques, the fiber probe tip includes an imaging element (e.g. lens) to capture reflected light from the tissue of interest. The fiber probe tip is adapted to maintain the positioning of the imaging element relative to the optical fiber to properly pass reflected light from the tissue sample to the optical fiber.

The fiber probe tip also employs an optical window on its distal end that is positioned relative to the imaging element. The optical window allows the reflected light from the tissue sample to pass through to the imaging element within the fiber probe tip. The optical window is located approximately one focal length away from the imaging element in one embodiment. This is so the reflected, scattered light from the tissue is properly captured when the fiber probe tip and its optical window are abutted to the tissue of interest. In the case of an angled-resolved LCI technique in the Fourier domain (fa/LCI) system, the fiber probe tip allows the maintenance of the tissue to be located approximately one focal length away from the imaging element so that the reflected, angular distribution of the reflected light is properly captured.

The fiber probe tip may employ different distal end designs to allow the fiber probe tip, and more particularly its optical window, to properly abut against the tissue of interest. The optical window should abut against the tissue of interest in order for the imaging element of the fiber probe tip to be located the proper distance away from the tissue of interest. The distal end of the fiber probe may be straight or angled to facilitate abutment to the tissue of interest. A suction device may also be employed on the distal end of the fiber probe tip to facilitate abutment to the tissue and provide stability. A separate channel path may be provided in the fiber probe tip to be used as a wash of the tissue and/or to provide vacuum assistance to assist in suction of the suction device to the tissue.

Designs are also provided to allow the fiber probe tip to either be fixed onto the fiber probe or removable. If removable, this allows the fiber probe tip to be disposed of after each endoscopic application to prevent washing and/or provide greater sterility. The design may include a locking system to lock the fiber probe tip in place during application. The fiber probe tip is then unlocked after use to then be disposed. If the locking system employs a channel in the sheath of the fiber probe tip, the fiber probe may be accessible from outside the fiber probe tip. Thus, a protective skirt may also be employed on the fiber probe tip. The skirt provides a method of covering the channel to prevent access to the fiber probe within the fiber probe tip and extending therefrom. The skirt can be designed to be retracted or coiled initially to allow the fiber probe tip to be easily and unobstructively attached to the fiber probe. The skirt can then be deployed after the fiber probe tip is attached and before endoscopic application begins.

The present invention is not limited to the embodiments presented here. Instead, any configuration which includes a probe tip with a rigid section which maintains the tissue under examination at a fixed distance from the fiber optic probe or its associated imaging, refractive, or diffractive elements can be seen as equivalent.

Those skilled in the art will appreciate the scope of the present invention and realize additional aspects thereof after reading the following detailed description of the preferred embodiments in association with the accompanying drawing figures.

### BRIEF DESCRIPTION OF THE DRAWINGS

FIG. 1 is a schematic of an exemplary low coherence interference (LCI) probe system employing an optical fiber probe;

FIG. 2 is an illustration of sample illumination and scattered light collection at the distal end of probe in the LCI system illustrated in FIG. 1;

FIG. 3 is an illustration of a probe tip that may be employed by the LCI system illustrated in FIG. 1;

FIG. 4 is an illustration of a cutaway view of a probe tip employing a fixed sheath in accordance with one embodiment of the invention;

FIG. 5 is an illustration of a solid view the probe tip illustrated in FIG. 4;

FIG. 6A is an illustration of a cutaway view of a probe tip employing a removable sheath in accordance with one embodiment of the invention;

FIG. 6B is an illustration of the probe tip illustrated in FIG. 6A, and employing an angled optical window in accordance with one embodiment of the invention;

FIG. 7 is an alternative illustration of a solid view of the probe tip illustrated in FIG. 6A;

FIG. 8 is an illustration of the probe tip illustrated in FIGS. 6A and 7, employing a sterile skirt in accordance with one embodiment of the invention;

FIG. 9 is an illustration of the probe tip illustrated in FIG. 8, with the sterile skirt deployed in accordance with one embodiment of the invention; and

FIG. 10 is an illustration of the probe tip illustrated in FIG. 9, employing a vacuum-assisted suction device to facilitate application of the probe tip to a tissue surface.

### DETAILED DESCRIPTION

The embodiments set forth below represent the necessary information to enable those skilled in the art to practice the invention and illustrate the best mode of practicing the invention. Upon reading the following description in light of the accompanying drawing figures, those skilled in the art will understand the concepts of the invention and will recognize applications of these concepts not particularly addressed herein. It should be understood that these concepts and applications fall within the scope of the disclosure and the accompanying claims.

In embodiments of the invention, the fiber probe tip includes a protective sheath over the optical fiber or bundle. The probe tip provides a sterile interface between the optical fiber and the tissue surface under examination during endoscopic applications. Because the fiber tip probe may be employed in optical spectroscopic techniques, the fiber probe tip includes an imaging element (e.g. lens) to capture reflected light from the tissue of interest. The fiber probe tip is adapted to maintain the positioning of the imaging element relative to the optical fiber to properly pass reflected light from the tissue sample to the optical fiber.

The fiber probe tip also employs an optical window on its distal end that is positioned relative to the imaging element. The optical window allows the reflected light from the tissue sample to pass through to the imaging element within the fiber probe tip. The optical window is located approximately one focal length away from the imaging element in one embodiment. This is so the reflected, scattered light from the tissue is properly captured when the fiber probe tip and its optical window are abutted to the tissue of interest. In the case of an angled-resolved LCI technique in the Fourier domain (fa/LCI) system, the fiber probe tip allows the maintenance of the tissue to be located approximately one focal length away from the imaging element so that the reflected, angular distribution of the reflected light is properly captured.

Advances of the present invention include at least four components: (1) inclusion of an imaging element in the fiber probe tip; (2) a removable/disposable section of the probe tip which maintains tissue position relative to the probe and/or imaging elements; (3) a sterile skirt or sheath to protect and maintain sterility of the remainder of probe; and/or (4) a suction device which may or may not be vacuum-assisted. Each of these components can be employed individually to a fiber probe tip in accordance with the invention or in any number and combination with each other.

In order to maintain these precise locations of the tissue of interest, imaging elements, and optical fibers, a probe tip should ideally be provided that contains rigid elements. These rigid elements preserve the spatial arrangements of the probe for proper capture of reflected light from the tissue. However, it may be impractical to encase an entire fiber optic probe in an enclosure. Instead, the distal portion of the tip can contain rigid elements with the remainder of the probe covered in a sheath, which prevents the probe from coming in contact with contaminating fluids. FIG. 4 illustrates a cutaway view of an exemplary fiber probe and a fiber probe tip 60 in this regard.

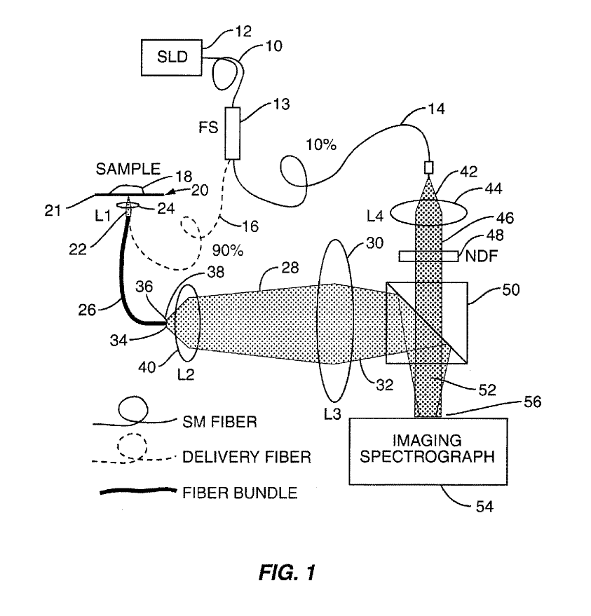

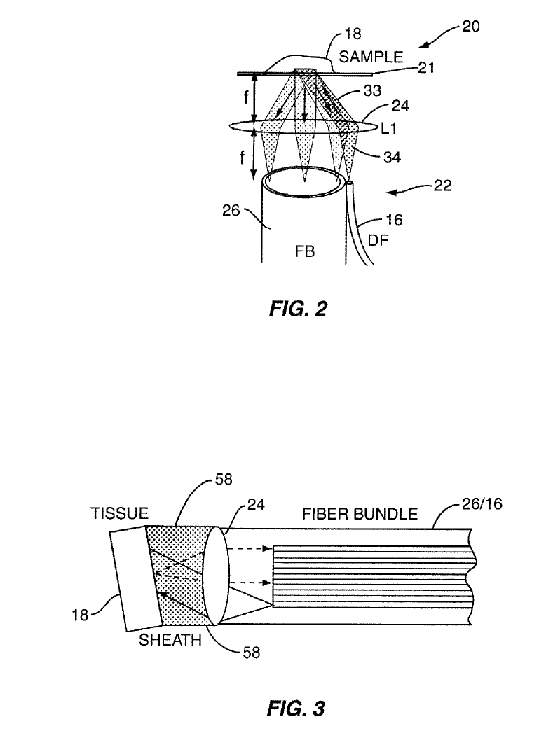

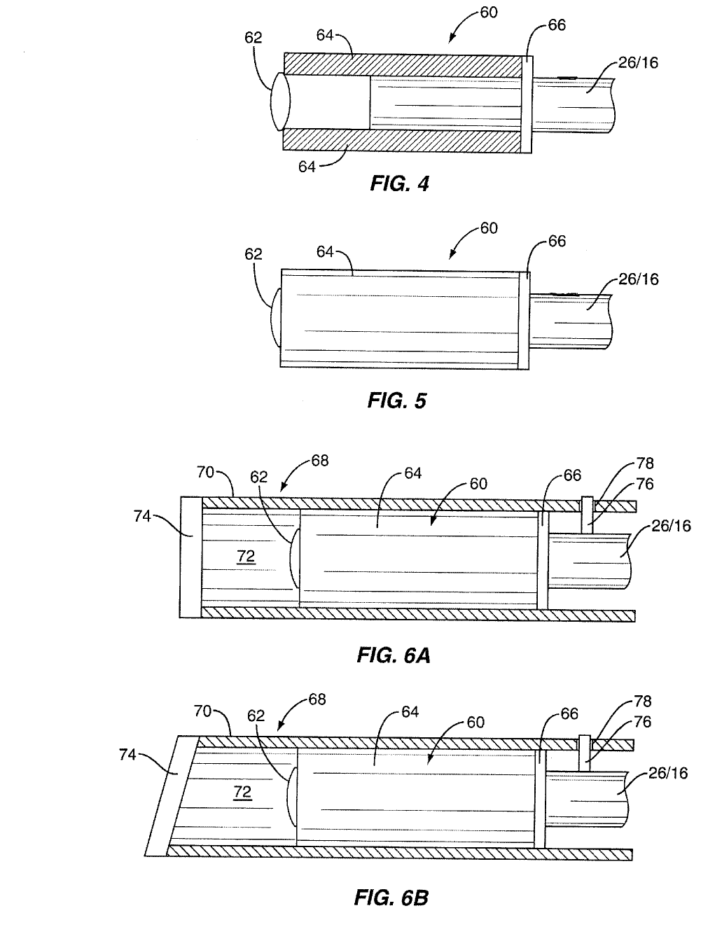

As illustrated in FIG. 4, a fiber probe tip 60 is provided in accordance with one embodiment of the invention. FIG. 5 illustrates the fiber probe tip 60 of FIG. 4, but in solid view. The fiber probe tip 60 is adapted to cover the distal end of an optical fiber used in an endoscopic imaging system. One example is the fiber optic probe employed in the fa/LCI system of FIGS. 1-3. If applied to this system, the distal ends of the delivery fiber 16 and fiber bundle 26 of the fiber probe system will be contained within the fiber probe tip 60, as illustrated in FIG. 4. Note however that the present invention is not limited to use in the fiber probe system of FIGS. 1-3.

One function of the probe tip 60 can be to create a fixed geometry between the optical fiber 16, 26, an imaging element, and the tissue under examination. Thus, a first component that can comprise the probe tip 60 is a means to locate an imaging element, such as a lens 62, relative to the fiber optic or bundle 16, 26. FIG. 1 shows a cutaway schematic of the use of a fixed sheath 64 comprised of a cylindrically-shaped outer wall having a hollow portion 65 placed over and surrounding the distal end of the fiber probe 16, 26 to position an imaging lens 62. In this embodiment, the fixed sheath 64, having a fixed length, is placed over the fiber bundle 16, 26 with a retaining ring 66 used to maintain the fixed distance between the fiber bundle 16, 26 and the lens 62. The fixed sheath 64, by being fixed, possesses a rigid construction to maintain the required positioning of the lens 62 relative to the fiber bundle 16, 26. The lens 62 is located on a distal end of the fixed sheath 64. The fixed sheath 64 can be affixed to the fiber probe 16, 26 with an adhesive, or can be attached to the retaining ring 66 using a flange or other locking mechanism. This configuration can be modified to include other types of optical elements or multiple optical elements (lenses, etc.).

If the probe tip 60 is employed in a fa/LCI system, like that illustrated in FIGS. 1-3, the lens 62 is placed approximately one focal length away from a fiber bundle 16, 26. This is required for the lens 62 to properly capture the reflected angular distribution of light from the tissue for analysis. In alternate embodiments, the lens 62 can be positioned such that an individual single or multimode fiber or an array of such fibers is maintained at the focus of the lens 62. In other embodiments, the imaging lens 62 can be positioned at other distances from the fiber optic element(s) 16, 26, which are different than the focal length of the lens 26.

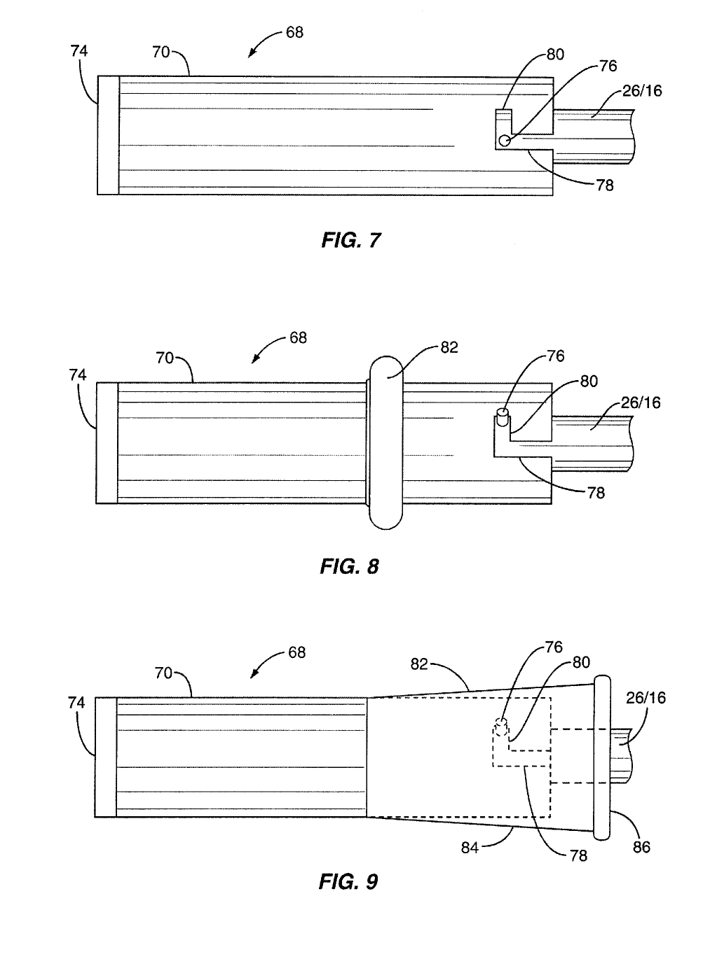

FIGS. 6A-7 illustrate an alternative embodiment of the fiber probe tip 60 incorporating a removable sheath member 68. The removable sheath member 68 is a structure that is adapted to receive the fixed sheath 64 of the fiber probe tip 60 to prevent the lens 62 and the fiber optics 16, 26 from being contaminated during endoscopic application. The removable member 68 is comprised of a cylindrical-shaped wall 70 containing a hollow portion 72 that receives and surrounds the fixed sheath 64 as part of the fiber probe tip 60. The distal end of the removable member 68 contains an optical window 74. The optical window 74 provides a path for reflected light from the tissue sample to pass back to the lens 62 in the fiber probe tip 62 to capture information about the tissue. The optical window 74 also flattens the tissue to provide for an even scan and to provide greater depth resolution accuracy. The optical window 74 can be made out of any material including glass, plastic, or comprise any other type of transparent material, including, but not limited to a membrane or other transparent material placed or stretched over the distal end of the disposable member 68. Anything that will transmit light can be used as the optical window 74.

The function of the optical window 74 is also to position the tissue relative to the lens 62 a proper distance from the tissue due to the rigid form of the cylindrical-shaped removable sheath 68. The abutment of the optical window 74 to the tissue surface provides a fixed distance between the tissue surface and the lens 26 in the fixed sheath 64. This may be necessary to properly capture reflected light from the tissue on the lens 62. Maintaining the relationships between the tissue (via the optical window 74) and the lens 62, and between the lens 62 and the fiber optics 16, 26 can be important in properly capturing reflected light from a tissue to analyze characteristics about its surface and/or underlying cell structures.

The optical window 74 may be perpendicular with respect to the longitudinal axis of the fiber probe tip 60 or may be slanted at an angle to allow better abutment of the optical window 74 to the tissue, as illustrated in FIG. 6B. Providing an angular configuration may help avoid reflection, which can obscure reflected scattered light captured at the optical window 74. But if the angle of the optical window 74 is slight, for example 0 to 20 degrees, and in a preferred embodiment, eight degrees, the lens 62 may still be able to properly capture the light and its angular distributions if the probe system is an angle-resolved system. If the angle of the optical window 74 will not allow the lens 62 to properly capture the angular distribution of the reflected, scattered light, the lens 62 can also be angled in the same or similar orientation to the optical window 74.

In an application of the fiber tip probe 60 designed for a fa/LCI system, the optical window 74 is designed on the disposable section 68 to be located approximately at the focal length of the lens 62. Providing the optical window 74 approximately one focal length away from the lens 62 allows the proper capture of the angular distributions of reflected light in the Fourier domain.

In alternative embodiments, the lens 26 may be integrated into the removable sheath member 68 as opposed to being integrated into the fixed sheath 64. Other alternative embodiments allow for different positioning of the optical window 74 relative to the lens 62.

In order to allow the removable sheath member 68 to be placed onto the fiber probe tip 60 and removed after endoscopic application, a locking mechanism may also be included. This prevents having to wash the fixed sheath 64 after each endoscopic application since the fixed sheath 64 and the lens 62 are not exposed when protected by the removable sheath member 68. In this regard, the removable sheath member 68 is first placed onto the fixed sheath 64 prior to application. Thereafter, it may be locked into place to prevent the removable member 68 from coming loose during application. After the fiber probe tip 60 is removed from endoscopic application, the removable member 68 can be unlocked and removed for disposal. In this manner, the fixed sheath 64 and exposed lens 62 are never exposed to the tissue and do not have to be washed. The lens 62, which may be one of the more expensive components of the fiber probe tip 60, does not have to be replaced or washed.

In the embodiments shown in FIGS. 6A-7, the removable sheath 68 is attached to the fiber bundle 16, 26 by sliding a locking pin 76 into a locking pin channel 78 in the removable member 68. Then, the removable member 68 is rotated with respect to the fixed sheath 64 to lock the removable member 68 in place. When it is desired to remove the removable member 68, such as after endoscopic application, the removable member 68 is rotated in the opposite direction from the locking rotation direction to allow the locking pin 76 to be removed from the locking pin channel 78. FIGS. 6A-6B illustrate the locking pin 76 engaged with the locking pin channel 78 in a cutaway view. FIG. 7 illustrates the locking pin channel 78 as it appears on the outside view of the removable sheath member 68. The locking pin channel 78 contains an angled channel portion 80 to allow the locking pin 76 to lock in place and provide resistance if the removable member 68 has a force applied to it opposite from the fiber optics 16, 26. The angled channel portion 80 is substantially a right angle with respect to the locking pin channel 78 in the illustrated embodiment. Note however that the locking pin channel 78 may provide an angled channel portion 80 at other angles other than a right angle. Alternative embodiments may also provide alternative means for locking the removable sheath 68 in place, including but not limited to a locking flange or ring mechanism.

While the removable sheath 68 described above will prevent direct contamination of the distal face of the fiber optics 16, 26, it is possible that fluids could penetrate through the locking pin channel 78 or to come in contact with the portion of the bundle 16, 26 which is not covered by the removable sheath member 68. For this reason, the probe tip 60 can be designed to additionally incorporate a deployable skirt 82 which will prevent such contamination. FIGS. 8 and 9 illustrate schematics views of the skirt 82 in an initial retracted or coiled and deployed or uncoiled position, respectively.

In the illustrated embodiment, the sterile skirt 82 is attached to the removable sheath member 68 at a point distal to the locking pin 76 and channel 78, 80. The skirt 82 can be composed of a plastic or latex material, suitable for preventing fluid from reaching the channel or bundle. The skirt 82 may be lubricated with any type of lubricant desired before being attached to the sheath member 68 and/or prior to endoscopic application. Prior to deployment, the skirt 82 may be coiled or otherwise collapsed to allow for facile manipulation of the locking pin 76 within the channel 78, 80, as illustrated in FIG. 8. Upon attachment of the removable sheath 68 to the fiber probe tip 60, the sterile skirt 82 can be deployed by rolling it down the sheath 68 toward the proximal end. FIG. 9 shows the deployment of the sterile skirt 82, wherein the skirt provides a protective outer covering 84 of the fiber probe 60 and/or the fiber optics 16, 26. The skirt 82 may also contains a rib 86 to maintain its deployment such that the rib 86 extends beyond the diameter of the fiber probe 60. In this manner, the skirt 82 can fill any accessory channel of an endoscope to prevent contaminants from reaching the fiber bundle 16, 26.

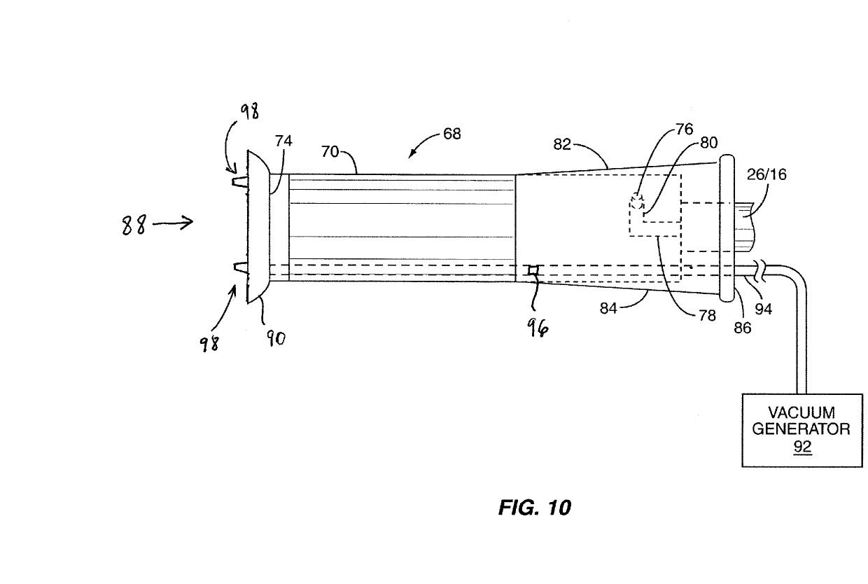

FIG. 10 illustrates an alternative embodiment of the fiber probe tip 60 of FIGS. 8 and 9, but with additional components to assist in the abutment of the optical window 74 to the tissue to maintain the distance between the tissue and the lens 62, and the stability between the optical window 74 and the tissue. As previously discussed, it may be important to ensure the abutment of the optical window 74 to the tissue to properly receive reflected light for analysis. In this regard, a suction device 88, such as a suction cup, may also be provided on the distal end of the removable sheath member 68 to provide suction between the tissue and the optical window 74 to assist in abutment. The suction device 88 may be useful in maintaining sufficient and stable contact between the optical window 74 and the tissue. The suction member 88 may comprise a circumference-shaped material 90 that is attached to the distal end of the removable member 86 and surrounds the optical window 74 so that reflected light is not obstructed. This material 90 may be any flexible material that can create a suction when pressed against a tissue surface. To provide further suction assistance, an external vacuum generator 92 may be employed and coupled to a vacuum or suction channel 94 located inside fiber probe tip 60. The vacuum generated by the vacuum generator 92 may be partially or fully assist in suction. A vacuum sensor or pressure transducer 96 may also be located within or coupled to the channel 94 to allow the detection of the pressure or vacuum at the optical window 74 to determine if a proper suction is being obtained between the tissue and the optical window 74 for proper endoscope examination. The vacuum or suction channel 94 may also be used as a tissue wash if coupled to an external wash. Grasping forcep 98 may also be provided that are controllable by the person applying the fiber probe 60 endoscopically to grasp the tissue to be examined to assist in the abutment of the tissue against the optical window 98.

The embodiments set forth above represent the necessary information to enable those skilled in the art to practice the invention and illustrate the best mode of practicing the invention. Upon reading the following description in light if the accompanying drawings figures, those skilled in the art will understand the concepts of the invention and will recognize applications of these concepts not particularly addressed herein. It should be understood that these concepts and applications fall within the scope of the disclosure. For example, the probe is not limited to a fiber optic probe or to use in any particular imaging system.

Those skilled in the art will recognize improvements and modifications to the preferred embodiments of the present invention. All such improvements and modifications are considered within the scope of the concepts disclosed herein and the claims that follow.

What is claimed is:

1. A probe tip for protecting an endoscopic probe during application, comprising:
a fixed sheath having a hollow portion adapted to fit over and surround a distal end of an endoscopic probe;
an imaging element affixed to the fixed sheath and adapted to receive and transmit a reflected light from a tissue as a result of a light projected onto the tissue by the endoscopic probe;
a removable sheath having a hollow portion and adapted to fit over and surround the fixed sheath; and
an optical window attached to a distal end of the removable sheath and adapted to abut against the tissue to receive and transmit the reflected light from the tissue to the imaging element as a result of a light projected onto the tissue by the endoscopic probe.

2. The probe tip of claim 1, wherein the imaging element is integrated into the hollow portion of the fixed sheath and located in a plane substantially perpendicular to the longitudinal axis of the fixed sheath.

3. The probe tip of claim 1, wherein the imaging element is located in the fixed sheath a fixed distance away from the distal end of the endoscope probe when the probe tip is placed over the distal end of the endoscopic probe.

4. The probe tip of claim 3, wherein the fixed distance is approximately one focus length of the imaging element.

5. The probe tip of claim 1, wherein the fixed sheath is cylindrical-shaped.

6. The probe tip of claim 1, further comprising a retaining ring that surrounds a proximal end of the fixed sheath to attach the fixed sheath to the distal end of the endoscopic probe.

7. The probe tip of claim 1, wherein the optical window is integrated into the hollow portion of the removable sheath and located in a plane substantially perpendicular to the longitudinal axis of the removable sheath.

8. The probe tip of claim 1, wherein the optical window is integrated into the hollow portion of the removable sheath and located in a plane angled to the perpendicular plane to the longitudinal axis of the removable sheath.

9. The probe tip of claim 8, wherein the imaging element is angled relative to the longitudinal axis of the fixed sheath.

10. The probe tip of claim 1, wherein the imaging element is angled relative to the longitudinal axis of the fixed sheath.

11. The probe tip of claim 1, wherein the optical window is located in the removable sheath a fixed distance away from the imaging element.

12. The probe tip of claim 10, wherein the fixed distance is approximately one focus length of the imaging element.

13. The probe of claim 11, wherein the imaging element is a lens adapted to capture an angular distribution of the reflected light in an optical Fourier transform of the reflected light.

14. The probe tip of claim 1, wherein the removable sheath is cylindrical-shaped.

15. The probe tip of claim 1, wherein the removable sheath is adapted to maintain sterility of the fixed sheath and imaging element during endoscopic application

16. The probe tip of claim 1, wherein the removable sheath contains a locking mechanism to lock the removable sheath to the fixed sheath.

17. The probe tip of claim 16, wherein the locking mechanism comprises a locking pin channel within the removable sheath adapted to receive a locking pin coupled to the endoscopic probe.

18. The probe tip of claim 17, wherein the locking pin channel is adapted to secure the locking pin to lock the removable sheath to the fixed sheath when the locking pin channel receives the locking pin and the removable sheath is rotated.

19. The probe tip of claim 1, further comprising an extendable skirt attached to the outside of the removable sheath and extendable towards the endoscopic probe to prevent contamination of the fixed sheath or the endoscopic probe or both the fixed sheath and the endoscopic probe.

20. The probe tip of claim 19, wherein an end of the extendable skirt not attached to the removable sheath contains a rib adapted to maintain deployment of the skirt when extended.

21. The probe tip of claim 19, wherein the extendable skirt is lubricated before endoscopic application.

22. The probe tip of claim 1, further comprising a suction member attached to the distal end of the removable sheath and surrounding the optical window to provide suction between the optical window and the tissue when the probe is applied endoscopically.

23. The probe tip of claim 22, further comprising a vacuum channel coupled to the suction member and adapted to generate a vacuum in the suction member when a vacuum generator coupled to the vacuum channel applies a vacuum to assist in providing suction of the optical window to the tissue.

24. The probe tip of claim 23, further comprising a pressure device coupled to the vacuum channel to sense the vacuum or pressure generated at the optical window

25. The probe tip of claim 1, further comprising a grasping forcep adapted to grasp the tissue to abut the tissue against the optical window during endoscopic application.

26. A system for probing a tissue endoscopically, comprising:
an imaging system having an endoscopic probe having a distal end adapted to direct a light towards a tissue to be examined; and
a fiber probe tip, comprising:
a fixed sheath having a hollow portion adapted to fit over and surround the distal end of the endoscopic probe;
an imaging element affixed to the fixed sheath and adapted to receive and transmit a reflected light from the tissue as a result of a light projected onto the tissue by the endoscopic probe;
a removable sheath having a hollow portion and adapted to fit over and surround the fixed sheath; and
an optical window attached to a distal end of the removable sheath and adapted to abut against the tissue to receive and transmit the reflected light from the tissue to the imaging element as a result of the light projected onto the tissue by the endoscopic probe.

27. The system of claim 26, wherein the endoscopic probe is a fiber-optic probe wherein the distal end of the fiber-optic probe comprises either an optic fiber or optic fiber bundle.

28. The system of claim 26, wherein the imaging system is an angled-resolved low coherence interferometry imaging system (a/LCI).

29. The system of claim 26, wherein the imaging element is located approximately one focus length away from the distal end of the endoscope probe when the probe tip is placed over the distal end of the endoscopic probe.

30. The system of claim 26, wherein the optical window is located in the removable sheath a focus length away from the imaging element.

31. The system of claim 29, wherein the imaging element is a lens adapted to capture the angular distribution of the reflected light in a Fourier transform of the reflected light.
